(19) United States
(12) Patent Application Publication
(10) Pub. No.: US 2018/0161124 A1
(43) Pub. Date: Jun. 14, 2018

(54) AXIAL STOP GAUGE AND JIG GUIDE FOR SURGICAL DRILL

(71) Applicant: Huwais IP Holding LLC, Jackson, MI (US)

(72) Inventor: Salah Huwais, Jackson, MI (US)

(21) Appl. No.: 15/575,831

(22) PCT Filed: May 20, 2016
(86) PCT No.: PCT/US16/33406

§ 371 Date: Nov. 21, 2017

Related U.S. Application Data
(60) Provisional application No. 62/164,799, filed on May 21, 2015.

(51) Int. Cl. A61C 1/08; A61B 17/17

(52) U.S. Cl. CPC A61C 1/084; A61B 2090/034; A61B 17/1757

(57) ABSTRACT

A surgical tool assembly for forming holes in bone at precise locations and to controlled depths. The drilling tool has a leading apical tip and a shank. The shank includes an annular groove at a predetermined distance from the apical tip. An adjustable stop gauge is coupled to the shank. The stop gauge includes an indexing adapter which telescopically supports a tubular collar. The collar includes an integrated impeller that accentuates irrigation of the treatment site during surgical procedures. The adjustable stop gauge may be used in combination with a guided surgery jig. The jig includes a guide bushing having a generally semi-cylindrical alignment valley adapted to receive the spinning collar of the stop gauge. The alignment valley includes an elevated internal abutment step having a semi-annular surface adapted to engage the collar when the apical tip reaches a predetermined penetration depth in the bone.

### CROSS REFERENCE TO RELATED APPLICATIONS

This application claims priority to Provisional Patent Application No. 62/164,799 filed May 21, 2015, the entire disclosure of which is hereby incorporated by reference and relied upon.

### BACKGROUND OF THE INVENTION

Field of the Invention. The invention relates generally to rotary drilling tools for forming an osteotomy or hole in bone or other cellular material to receive an implant or other fixation device, and more specifically toward a novel stop gauge that prevents penetration of the drilling tool beyond a predetermined depth, as well as to a novel guided surgery jig used in combinations therewith.

Description of Related Art. An implant is a medical device manufactured to replace a missing biological structure, to support a damaged biological structure, or to enhance an existing biological structure. Bone implants may be found throughout the human skeletal system, including dental implants in a jaw bone to replace a lost or damaged tooth, vertebral implants used to secure cages, joint implants to replace a damaged joints such as hips and knees, and reinforcement implants installed to repair fractures and remediate other deficiencies, to name but a few. The placement of an implant often requires a preparation into the bone using either hand osteotomes or precision drills with highly regulated speed to prevent burning or pressure necrosis of the bone. After a variable amount of time to allow the bone to grow on to the surface of the implant (or in some cases to a fixture portion of an implant), sufficient healing will enable a patient to start rehabilitation therapy or return to normal use or perhaps the placement of a restoration or other attachment feature.

In the example of a dental implant, preparation of a hole or osteotomy is required to receive a bone implant. The depth of an osteotomy is determined by the amount of axial movement that the clinician imparts on a drilling tool as he or she inserts the drilling tool into the bone tissue. If the depth of the bore is too long, it can puncture the sinus cavity in the maxillary, or the mandibular canal (which contains nerves) in the mandible. Likewise, the roots of adjacent teeth also can be adversely affected by an improperly sized osteotomy.

To ensure that a drilling tool is inserted into the bone to a known depth, the drilling tool may contain markings that signify specific depths. For example, a drilling tool may have bands of etched markings that indicate the bore depth at several locations. The use of these visual markers is, of course, limited to the clinician's ability to see the mark as the drilling tool is being inserted into the patient's mouth. Accordingly, the clinician is required to keep his or her visual attention on the depth marker as he or she slowly proceeds with the axial movement that causes the drilling tool to be inserted deeper and deeper into the bone. Visibility in such cases can be obscured by irrigation fluid and tools and other obstructions, making the traditional visual markers sometimes difficult to use.

The prior art discloses various types of stop elements that prohibit insertion of a drill or bur into the bone tissue beyond a predetermined depth. The methods employed by these prior are schemes are either difficult/cumbersome to use, or are expensive to produce. A few notable examples are described below.

U.S. Publication No. 2007/0099150 to Daniele discloses a depth stop collar for a dental drill. The shank of the drill has a series of grooves. Pawls at the top of the stop collar selectively engage the grooves in the shank to set the drilling depth. Drilling depth is adjusted by moving the stop collar up or down along the drill shank.

German patent document DE3800482 to List teaches a depth stop for a surgical drill. A series of annular ribs are formed along the drill shank. A stop collar fitted with a spring and ball locking mechanism sequentially snaps into the annular ribs to set the drilling depth.

U.S. Pat. No. 7,569,058 to Ralph discloses an adjustable depth stop for a surgical device used to form pre-threaded holes in bone. A series of annular ribs are formed along the length of the tap shank. A stop collar fitted with flexible pawls sequentially snaps into the annular ribs to set the tap depth. A screw-on locking cap threads over the flexible pawls to secure them in an adjusted position.

Common disadvantages perceived among the prior art are many, and include lack of ability to be installed on and removed from any drilling tool. Rather, in each case a specially manufactured drilling tool is required. Another common disadvantage is that multiple grooves must be formed in the tool shank. For high-speed applications, the multiple grooves risk weakening the shank with multiple stress-concentrating nodes that invite unwanted vibrations in use. The multiple grooves also add to manufacturing expense. And furthermore, each groove in the shank represents a hard-to-clean location for post-operative sterilization prior to re-use. Multiple grooves in the tool shank compound this concern, resulting in increased time and effort required during the customary sterilization and cleaning processes. Still further disadvantages of the prior art depth-stop concepts relate to the overall lack of suitability for retrofit use across a wide range of drilling tools marketed by different manufacturers. And yet further, none of the prior art depth-stop concepts are well-suited for use with the growing demand for guided surgery applications.

Korean patent document KR20060096849 to Hsieh discloses a guided surgery system in which a mouth jig has a guide feature to provide location and orientation control. Hsieh teaches the diameter of the guide feature can be reduced by adhering an additional magnetic guide bushing. However, the Hsieh system is not coordinated for use with a depth-stop feature, thereby making it difficult or cumbersome to utilize depth control in combination with guided surgery.

There is therefore a need in the art for an improved stop element that prohibits insertion of a surgical drilling tool or bur into the bone tissue beyond a predetermined depth, and which can be used conveniently in combination with a jig for guided surgery.

### BRIEF SUMMARY OF THE INVENTION

According to a first aspect of the present invention, an adjustable stop gauge is provided for a bone drilling tool of the type having a body section and a shank joined in end-to-end fashion. The adjustable stop gauge comprises a tubular collar adapted to partially surround the body of a bone drilling tool. The collar defines a stop ring that is adapted to limit over-penetration of an apical tip of the drilling tool into bone. An indexing adapter is connectable to the drilling tool and moveably supports the collar. The indexing adapter is configured to selectively locate the stop ring in any one of a plurality of longitudinal stations, wherein each station represents a different penetration depth of the drilling tool in the bone.

The indexing adapter can be easily installed on and removed from any drilling tool. The indexing adapter enables an adjustable position collar without the prior art requirement of forming multiple grooves in the shank of the drilling tool, thereby maintaining the strength of the drilling tool and reducing the tendency for unwanted vibrations in use. Furthermore, the indexing adapter reduces the need for expensive additional manufacturing operations on the drilling tool in order to accommodate an adjustable collar. And still further, the indexing adapter decreases the effort required post-operatively during the customary sterilization and cleaning processes, as compared with prior art style adjustable position depth stop systems.

According to a second aspect of the present invention, a stop gauge is provided for a bone drilling tool of the type having a body section and a shank joined in end-to-end fashion. The stop gauge comprises a tubular collar that is centered about a central axis. The collar is adapted to surround, at least partially, the working end or body of a drilling tool. The collar has a stop ring that is adapted to limit over-penetration of an apical tip of the drilling tool into the bone. The collar includes a plurality of vane slots that are configured to permit the pass-through of irrigating fluid.

The vane slots permit irrigating fluid to better wash over the osteotomy site, thereby allowing for better heat management at the treatment site. When an optional auto-grafting rotary osteotome is used as the hole-forming tool, the generous flow of irrigating fluid through the vane slots allows the osteotome to generate hydrodynamic effects which substantially enhance the hole formation procedure.

According to a third aspect of the present invention, a dental tool assembly is provided for forming a hole of predetermined depth in bone. The assembly comprises a tubular collar adapted to partially surround the body of a bone drilling tool. The collar defines a stop ring that is adapted to limit over-penetration of an apical tip of the drilling tool into bone. A jig is configured to be secured relative to a target drilling location. The jig has a guide bushing that provides a laterally open alignment valley adapted to receive the collar of the stop gauge. The alignment valley includes an internal abutment step which is adapted to engage the stop ring of the collar when the apical tip of a drilling tool has reached a predetermined penetration limit in the bone.

The alignment valley provides maximum access and visibility into an edentulous jaw site for the surgeon. The laterally open configuration allows substantially increased irrigation capacity to the osteotomy, as compared with prior art designs. The open configuration of the alignment valley allows the bone to freely expand laterally, such as when used in combination with a rotary expanding osteotome. Relatively long-length drilling tools can be navigated laterally into position. The abutment step establishes a stable, elevated surface configured to engage the spinning stop ring of the collar when the drilling tool has reached the desired drilling depth. Unlike the often imperfect surface of a patient's natural skin or exposed bone, the abutment step can afford the surgeon certain and immediate haptic feedback when the desired drilling depth has been achieved. In cases where the collar is made from a polymeric material, the abutment step may help avoid abrasion or distortion, thus extending the operating life of the collar and perhaps enabling re-use of the collar in one or more future surgical applications.

### BRIEF DESCRIPTION OF THE SEVERAL VIEWS OF THE DRAWINGS

These and other features and advantages of the present invention will become more readily appreciated when considered in connection with the following detailed description and appended drawings, wherein:

FIG. 1 depicts an exemplary application of the present invention at an edentulous jaw site that needs expansion to receive an implant;

FIG. 2 is a view as in FIG. 1, but showing the osteotomy in the process of being prepared with a drilling tool fitted with a stop gauge according to the present invention;

FIG. 3 is a view as in FIG. 2 showing the drilling tool at full depth as limited by the stop gauge and the concurrent application of irrigating fluid;

FIG. 4 shows the resulting fully prepared osteotomy ready to receive an implant;

FIG. 5 is a view as in FIG. 4 in which an installed implant is poised to receive an abutment or base for subsequent prosthetic (not shown);

FIG. 6 is an exploded view of a drilling tool according to one embodiment of the invention, in which an auto-grafting rotary osteotome is fitted with an adjustable stop gauge;

FIG. 7 is a perspective view of a drilling tool with adjustable stop gauge shown in a lowermost adjusted position or longitudinal station in solid lines, and in an uppermost adjusted position or longitudinal station in phantom;

FIG. 8 is a cross-sectional view taken generally along lines 8-8 in FIG. 7;

FIG. 9 is a cross-sectional view as in FIG. 8 but depicting the irrigation fluid passing capability provided by vane slots in the stop gauge;

FIG. 10 is a cross-sectional view taken generally along lines 10-10 in FIG. 9;

FIG. 11 is an enlarged cross-section through one of the vane slots as indicated by the area circumscribed at 11 in FIG. 10, and showing irrigating fluid being impelled through a vane slot as the stop gauge and the drilling tool rotate in a clockwise direction;

FIG. 12 is a view as in FIG. 11, but showing irrigating fluid being impelled through a vane slot as the stop gauge and drilling tool rotate in a counter-clockwise direction;

FIG. 13 is a view as in FIG. 2 showing an osteotomy in the process of being prepared with an auto-grafting rotary osteotome fitted with a stop gauge according to the present invention, and wherein a guided surgery jig is used to provide alignment assistance;

FIG. 14 is a view as in FIG. 13 showing the rotary osteotome at full depth as limited by the stop gauge and the concurrent application of irrigating fluid;

FIG. 15 is a cross-sectional view as taken generally along lines 15-15 in FIG. 14;

FIG. 16 is a cross-sectional view as in FIG. 15, but showing a different drilling depth due to the adjustable collar being positioned in a different longitudinal station;

FIG. 17 is a simplified depiction of a human skeleton highlighting some examples of areas in which the present invention might be effectively applied;

FIG. 18 is an enlarged view of a human vertebrae; and

FIG. 19 is a view of the vertebrae as in FIG. 18 shown in cross-section with a combined rotary osteotome and depth stop gauge according to one embodiment of this invention disposed to enlarge an osteotomy for the purpose of receiving a fixation screw or other implant device.

### DETAILED DESCRIPTION OF THE INVENTION

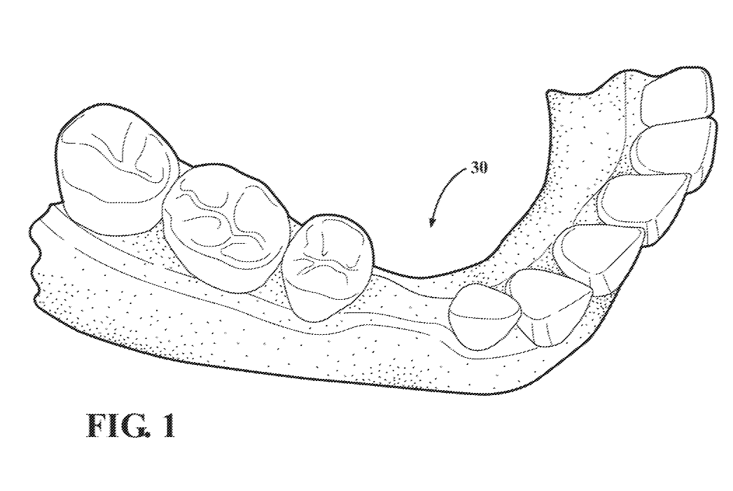

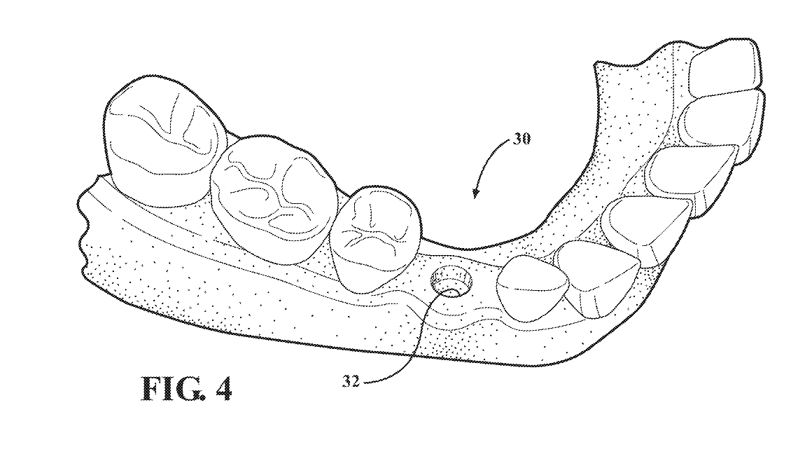

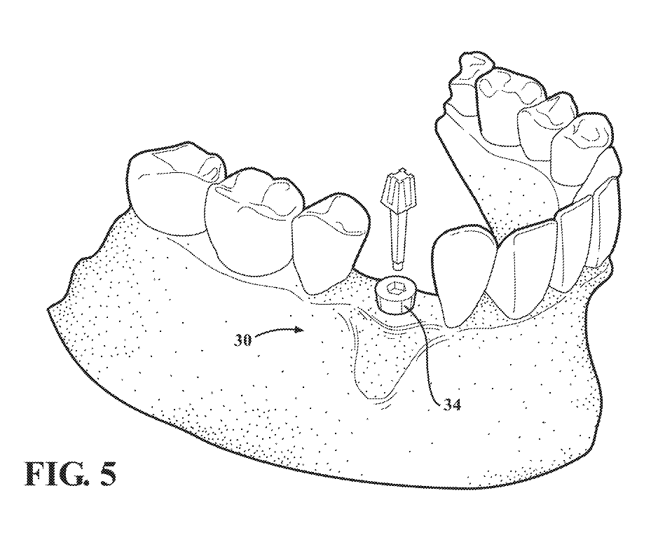

Referring to the figures, wherein like numerals indicate like or corresponding parts throughout the several views, FIGS. 1-5 show the exemplary periodontal context of an edentulous jaw site 30, in which an osteotomy 32 must be prepared in order to receive an implant 34. In FIG. 1, the edentulous jaw site 30 is shown in the pre-operative condition. One method of preparing an osteotomy 32 is to drill a hole using a traditional drilling-type dental bur that bores into and excavates the host bone material. Another method is described in U.S. Pat. No. 9,028,253 issued May 12, 2015 to Huwais, the entire disclosure of which is hereby incorporated by reference in jurisdictions that recognize incorporation by reference. According to the method of U.S. Pat. No. 9,028,253, a pilot hole is first bored into the recipient bone at the edentulous jaw site 30. The small bored pilot hole is then expanded using one or more (i.e., progressively larger) high-speed rotary osteotomes 36 fitted in a hand-held surgical drill motor 38. The rotary osteotome 36 is designed to auto-graft the host bone material directly into the sidewalls of the osteotomy 32 while forcibly expanding the osteotomy 32 using modulated pressure combined with copious irrigation 39, resulting in a smooth, highly densified osteotomy 32 capable of providing high initial stability for a subsequently placed implant 34 or other fixture device. However, it will be appreciated that the inventive features of this present invention are not exclusively limited to use with the rotary osteotome 36 like that depicted in the drawings. Nevertheless, the present invention is well-adapted for use with the high-speed rotary condensing osteotome 36, and is therefore referenced as a preferred example herein.

The rotary osteotome 36 is described in U.S. Pat. No. 9,326,778 issued May 3, 2016, and also in WO 2015/138842 published Sep. 17, 2015, both to Huwais, the entire disclosures of which are hereby incorporated by reference in jurisdictions that recognize incorporation by reference. Generally stated, the auto-grafting osteotome 36 includes a shank 40 and a working end or body 42. The shank 40 is basically an elongated cylindrical shaft that establishes a longitudinal axis of rotation A for the rotary osteotome 36 when driven at high speed (e.g., greater than 200 rpm; typically in the range of 800-1500 rpm) by the drill motor 38. A drill motor engaging interface 44 is formed at the distal upper end of the shank 40 for connection to the drill motor 38. Of course, the particular configuration of the interface 44 may vary depending on the type of drill motor 38 used, and in some cases may even be merely a smooth portion of the shank shaft against which the jaws of a collet grip by friction alone. An annular groove 45 is disposed at a predetermined intermediate axial location along the shank 40. The groove 45 is preferably shallow, with relatively square inset corners. The longitudinal length (i.e., width) of the groove 45 may be in the range of about 10% to 100% of the diameter of the shank 40, although widths of greater or lesser dimensions are possible.

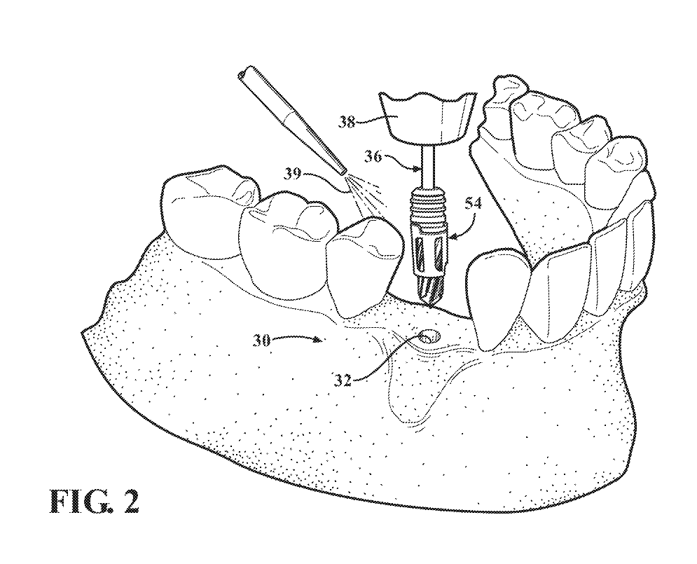

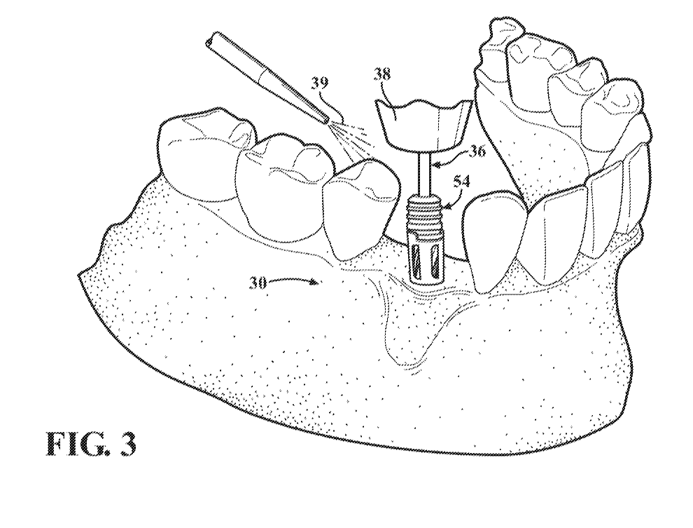

The body 42 of the osteotome 36 joins to the shank 40 at a transition 46, which may be formed with a tapered or domed shape. The angle or pitch of the transition 46 may be described by a transition angle measured relative to the longitudinal axis A. The transition 46 normally helps spread the irrigating fluid something like an umbrella as the surgeon irrigates with water (or saline, etc.) during use. Irrigation of the osteotomy site 32, as depicted at 39 in FIGS. 2 and 3, is especially important when using an auto-grafting type rotary osteotome 36 to facilitate its hydrodynamic affects and to manage heat.

The working end or body 42 of the osteotome 36 has conically tapered profile decreasing from a maximum diameter adjacent the shank 40 to a minimum diameter adjacent an apical end 48. The apical end 48 is thus remote from the shank 40, with the aforementioned groove 45 being located along the shank 40 at a predetermined distance from the apical tip 48 for reasons that will be described. The working length or effective length of the body 42 is proportionally related to its taper angle and to the size and number of osteotomes in a surgical kit in cases where the osteotomy 32 is formed by a sequence of progressively larger osteotomes 36. Preferably, all osteotomes 36 in a kit will have the same taper angle, and the diameter at the upper end of the body 42 for one osteotome is approximately equal to the diameter adjacent the apical end of the body 42 for the next larger size osteotome.

The apical end 48 may include one or more lips 50. A plurality of grooves or flutes 52 are disposed about the body 42. The flutes 52 are preferably, but not necessarily, equally circumferentially arranged about the body 42. A rib or land is formed between adjacent flutes 52, in alternating fashion. Thus, a four-flute 52 osteotome 36 will have four interposed lands, a ten-flute 52 osteotome 36 will have ten interleaved lands, and so forth. Each land forms a working edge. Depending on the rotational direction of the osteotome 36, the working edge either functions to cut bone or condense bone. That is, when the osteotome 36 is rotated in the cutting direction, the working edges slice and excavate bone (or other host material). However, when the osteotome 36 is rotated in the condensing (non-cutting) direction and pushed into the osteotomy 32 with modulating pressure, the working edges compress and radially displace bone with little to no cutting whatsoever. This compression and radial displacement is exhibited as gentle pushing of the osseous structure laterally outwardly in a condensation mechanism.

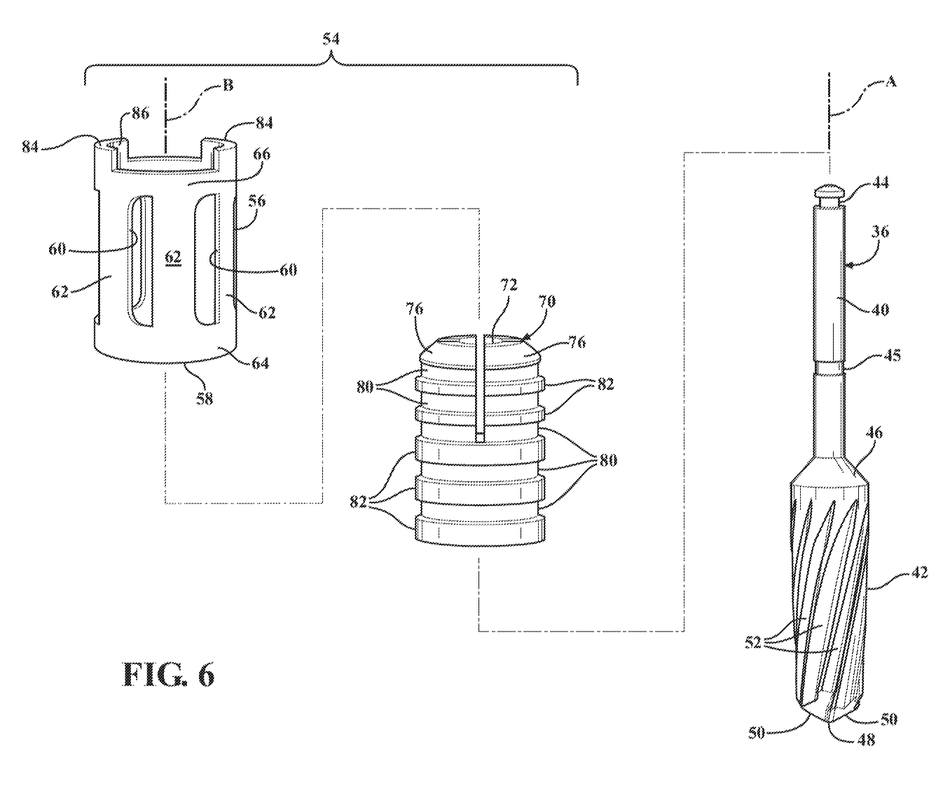

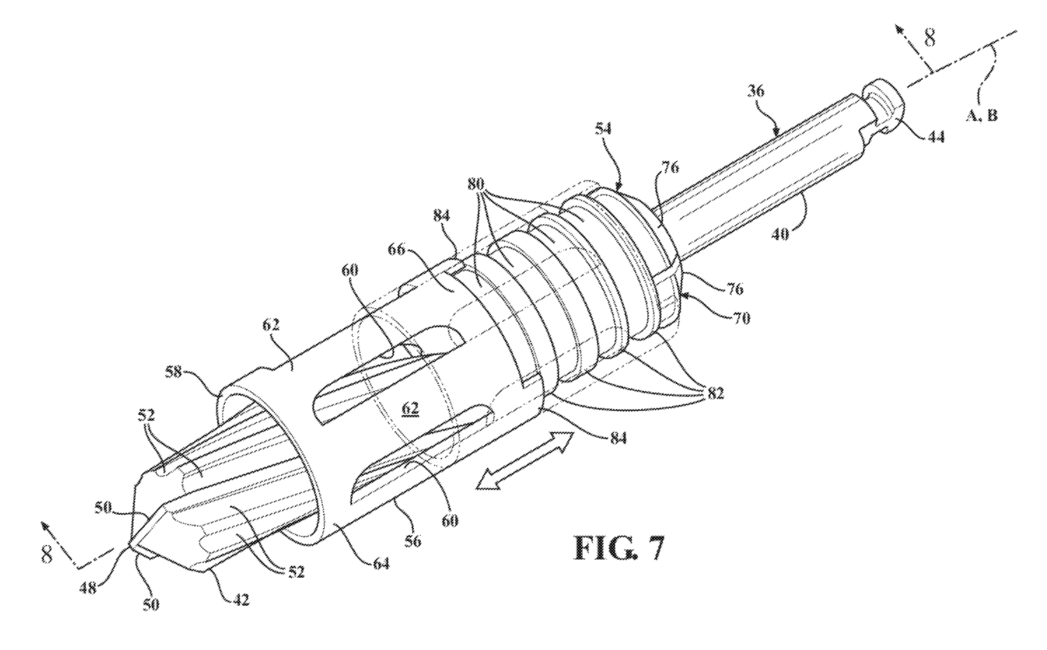

To ensure that the apical end 48 of the rotary osteotome 36 (or the tip of a traditional drilling bur or other boring tool) does not exceed a desired depth in the bone, an axial stop gauge, generally indicated at 54, is provided. The stop gauge 54 is shown exploded in FIG. 6 and assembled together with a rotary osteotome 36 in FIG. 7. The stop gauge 54 comprises a tubular collar, generally indicated at 56, that is centered about a central axis B. The central axis B coincides with the longitudinal axis A of the shank 40 when the two are assembled together as a unit. The collar 56 has a generally constant exterior diameter. An annular stop ring 58 is formed at the lowermost end of the collar 56. The stop ring 58 is preferably smooth and lies perpendicular to the central axis B. However, in some contemplated embodiments the surface of the stop ring 58 may be textured or crenelated.

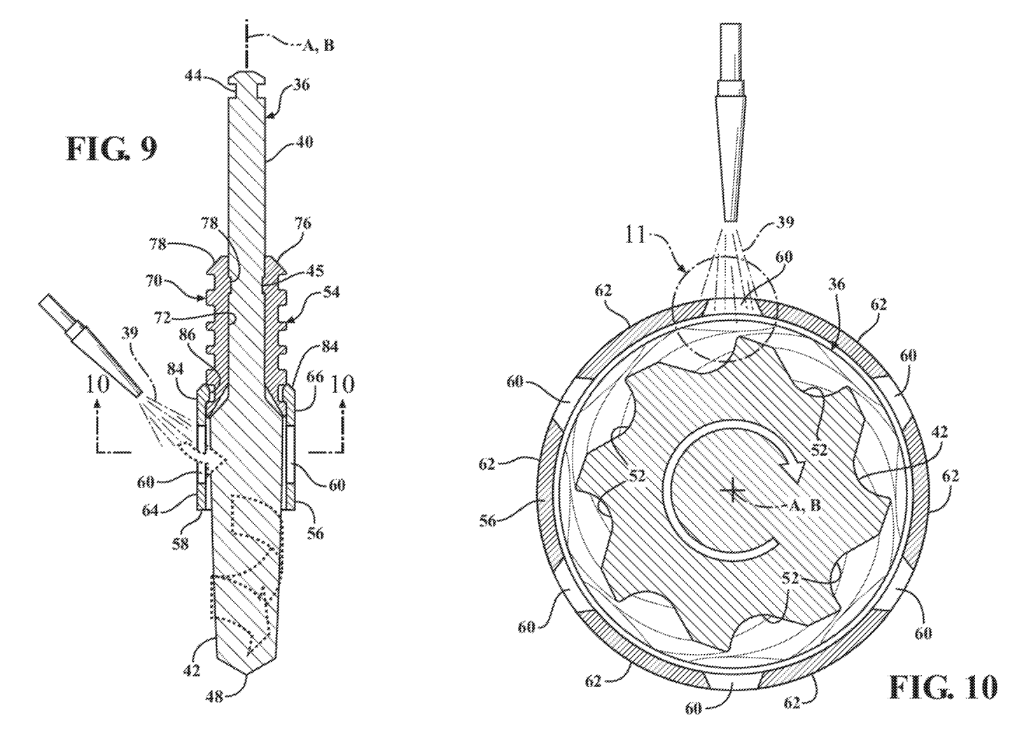

The collar 56 may, optionally, include a plurality of vane slots 60 configured to permit irrigating fluid 39 to readily pass-through to reach the body section 42 of the osteotome 36. The vane slots 60, therefore, allow the irrigating fluid 39 to better wash over the osteotomy site 32, thereby allowing for better heat management at the treatment site. As illustrated by the broken directional arrows in FIG. 9, irrigating fluid 39 directed toward the collar 56 passes laterally through the slots 60, allowing the fluid 39 to engage the drilling tool 36 and then be pulled by its flutes 52 in a downward spiral (into the osteotomy 32). When an auto-grafting rotary osteotome 36 is used as the hole-forming tool, the generous flow of irrigating fluid 39 through the vane slots 60 allows the osteotome 36 to generate hydrodynamic effects which, as described in the aforementioned WO 2015/138842, substantially enhance the hole formation procedure.

The vane slots 60 may, optionally, be configured as an integral impeller to accelerate the radially inward flow of water. In this configuration, the rotating motion of the collar 56 (synchronously locked to the rotating osteotome 36 through friction) is used as an energy source, together with the shape or configuration of the vane slots 60, to facilitate movement of the irrigating fluid inwardly toward the osteotome 36. Each pair of adjacent the vane slots 60 may be seen as being circumferentially separated by a respective longitudinally extending blade 62. The blades 62 terminate at the lower end of the collar 56, i.e., near the stop ring 58, at an annular lower cuff 64. Said another way, the lower cuff 64 is the region of the collar 56 between the stop ring 58 and the vane slots 60. Similarly, an annular upper cuff 66 is formed by the region of the collar 56 between its upper end and the vane slots 60. The blades 62 thus extend between the upper 66 and lower 64 cuffs forming a ventilated cage-like structure.

In the illustrated embodiment, the vane slots 60 are spaced from one another in equal circumferential increments about the collar 56, and the blades 62 are each of generally equal width forming a symmetrical appearance. The vane slots 60 and the interposed blades 62 respectively extend in generally straight axial paths parallel to one another and parallel to the central axis B. However, in a contemplated alternative embodiment, the vane slots 60 and blades 62 may be spiraled or slanted in their arrangement around the collar 56 to promote irrigation flow 39 if desired. Indeed, the vane slots 60 need not even be slots per se, but could in fact be designed as holes of round or other geometric shape that permit the pass-through of irrigating fluid (with or without an impeller effect). The number and/or relative sizes of the vane slots 60 may depend on the exterior diameter of the collar 56 and the width of the intervening blades 62. In the examples shown in FIGS. 6, 7 and 10, six vane slots 60 (and six blades 62) are arranged in equal circumferential increments about the collar 56. Furthermore, the number or size of vane slots 60 (or blades 62) may be determined considering how much irrigating fluid is required for the body section 42 of the osteotome 36 and/or the degree to which column strength of the collar 56 is affected.

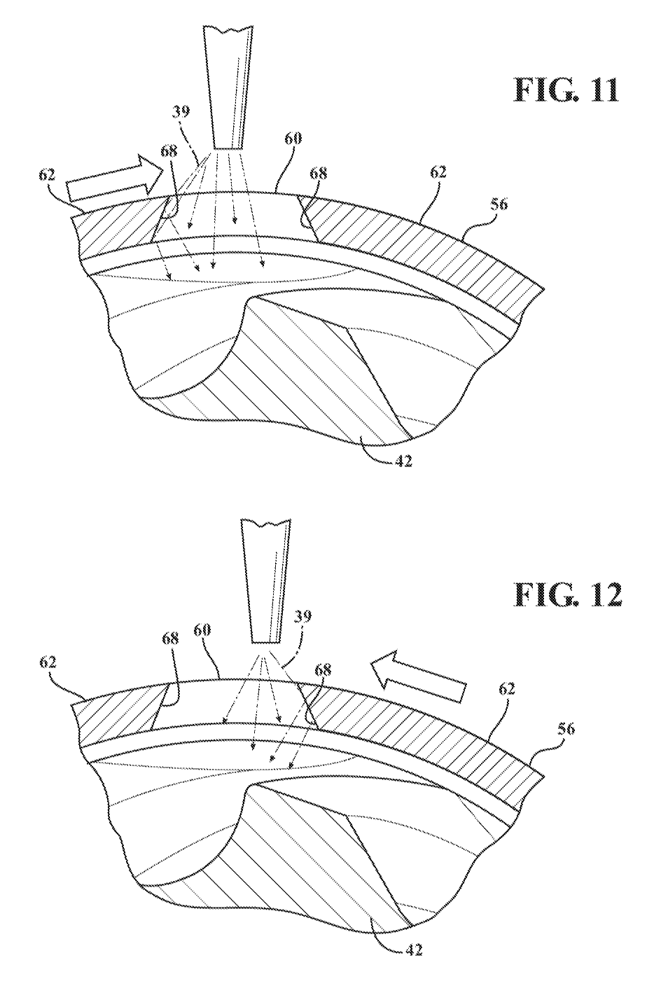

Turning to FIGS. 11-12, one exemplary embodiment is illustrated as a means by which to accomplish the impeller function the shape of the blades 62. Each blade 62 can be viewed as having a pair of longitudinally extending edges 68. The edges 68 define the respective boundaries with the adjacent the vane slots 60. At least one of the edges 68 of each blade 62 may be canted or sloped like a chisel to impel irrigating fluid through the adjoining vane slot 60 along a radially inward vector when the stop gauge 54 is rotated about its central axis B. Preferably, both edges 68 of the blade 62 are canted, in opposing directions, so that irrigating fluid will be propelled inwardly through the vane slots 60 when the stop gauge 54 is rotated about the central axis B in either rotary direction. FIG. 11 shows that irrigating fluid 39 is impelled inwardly toward the body section 42 of the osteotome 36 when the stop gauge 54 is rotated clockwise. To be specific, the left-side edge 68 is slanted inward so that irrigating fluid 39 that comes in contact with that edge 68 is urged or pumped inwardly. Conversely, FIG. 12 shows that irrigating fluid 39 will be propelled inwardly through the vane slot 60 when the stop gauge 54 is rotated counter-clockwise.

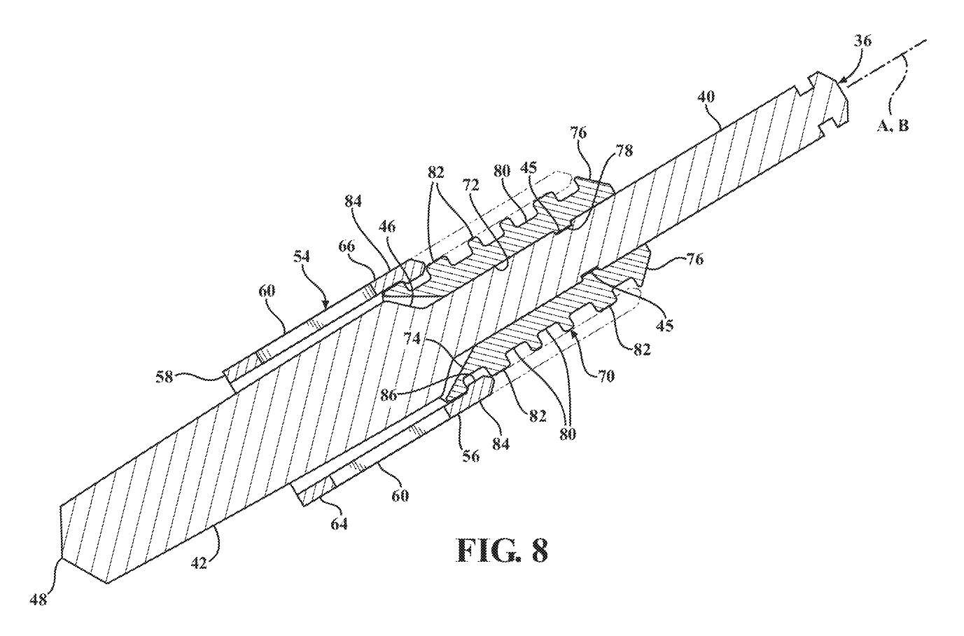

The stop gauge 54 may be of a fixed length design, i.e., so that only one predetermined drilling depth is possible, or alternatively may include an indexing adapter, generally indicated at 70, that allows the collar 56 to be moved to various pre-selected longitudinal stations relative to the osteotome 36. In this manner, the stop ring 58 can be set or re-set, at the time of use, at different heights relative to the apical end 48 of the osteotome 36, thus achieving different pre-determined drilling depths into the bone. For example, in the illustrated embodiment five longitudinal stations are established, respectively corresponding to drilling depth limits of 6, 8, 10, 11.5 and 13 mm. That is to say, when the collar 56 is moved to the first longitudinal station along the indexing adapter 70, the axial distance between the apical end 48 and the stop ring 58 is 6 mm. In FIGS. 7-9, the collar 56 is shown in solid lines positioned in this first longitudinal station. When the collar 56 is moved to the second longitudinal station along the indexing adapter 70, the axial distance between the apical end 48 and the stop ring 58 is 8 mm. When the collar 56 is moved to the third/middle longitudinal station along the indexing adapter 70, the axial distance between the apical end 48 and the stop ring 58 is 10 mm. When the collar 56 is moved to the fourth longitudinal station along the indexing adapter 70, the axial distance between the apical end 48 and the stop ring 58 is 11.5 mm. And when the collar 56 is moved to the fifth/last longitudinal station along the indexing adapter 70, the axial distance between the apical end 48 and the stop ring 58 is 13 mm. In FIGS. 7 and 8, the collar 56 is shown in broken or phantom lines positioned in this fifth/last longitudinal station. Of course, the indexing adapter 70 can be configured with more or less longitudinal stations, and the predetermined distances between apical end 48 and stop ring 58 can be designed to suit different needs or applications.

As shown in the cross-sectional view of FIG. 8, the indexing adapter 70 locks onto the shank 40 via its groove 45. In this manner, the indexing adapter 70 is securely positioned between the osteotome 36 and the collar 56. (In the aforementioned case where the stop gauge 54 is of a fixed length design, the collar 56 could be configured to directly connect to the groove 45 without an intermediate indexing adapter 70.) In the illustrated examples, the indexing adapter 70 has a generally cylindrical or barrel-like shape centered along the central axis B. A central bore 72 passes through the indexing adapter 70 and is sized to snugly surround the shank 40 with no discernable play between the two. The lower end of the central bore 72 opens into a conically widening throat 74. The funnel-like conical pitch of the throat 74 is formed at a throat angle relative to the central axis B, as shown in FIG. 8. In one embodiment, the throat angle may be intentionally designed smaller than the transition angle, which provides some advantages when design-paired with the axial location of the transition 42 on the osteotome 36. For example, the throat 74 of the indexing adapter 70 and the transition 46 of the osteotome 36 may be designed to meet along a circular line of contact, which may improve the locking force and/or rotational stability between the indexing adapter 70 and the osteotome 36, particularly for larger diameter osteotomes 36. The circular line of contact may also help establish a seal between the indexing adapter 70 and the osteotome 36 so that debris will be less inclined to accumulate behind their interface which could potentially build up pressure and urge a disconnection of the indexing adapter 70 from the groove 45.

The top end of the indexing adapter 70 includes at least one, and preferably a plurality of, cantilever locking segments 76. In the illustrated examples, the indexing adapter 70 is formed with four locking segments 76. The locking segments 76 could be formed by cutting one or more narrow radial slits into the top of the indexing adapter 70. Each locking segment 76 includes a spur 78 that extends inwardly from the central bore 72 and engages within the annular groove 45 of the shank 40, as best seen in FIG. 8. In this manner, the indexing adapter 70 self-locks to the shank 40 of the osteotome 36 when the indexing adapter 70 is slid into position and its one or more spurs 78 register with groove 45. The locking segments 76 may each be formed with a chamfered nose to help distribute the flow of irrigating fluid 39 in use.

The aforementioned longitudinal stations of the collar 56 are established by annular channels 80 disposed about the external surface of the indexing adapter 70. Each adjacent pair of the channels 80 is separated by a respective annular rib 82. Depth number indicia may be disposed in or near the channels 80 to indicate a distance between the stop ring 58 and the apical end 48 corresponding to each longitudinal station. For example, using the previous exemplary pre-set drilling depths, the number “6” could be visibly embossed inside the first annular channel 80; the number “8” in the second annular channel 80; the number “10” in the third annular channel 80; the number “11.5” in the fourth annular channel 80; and the number “13” in the fifth/last annular channel 80.

One or more fingers 84 extend from the upper end of the collar 56, each carrying an inwardly extending prong or barb 86 designed to seat within a selected one of the annular channels 80. The annular channels 80 are each the same width which corresponds to the width of the barbs 86, and are therefore adapted to selectively receive the inwardly extending barbs 86 of the collar 56 as the collar 56 is moved from one longitudinal station to another. (Although, the width of the ribs 82 will vary depending on the predetermined spacing between the longitudinal stations.) The barbs 86 may be chamfered with camming faces to facilitate movement between the annular channels 80 as a user moves the collar 56 from one longitudinal station to another in setting and re-setting the depth stop. In this manner, the fingers 84 resiliently flex as the barbs 86 move into and out of registry with the annular channels 80 using moderately applied external force, and yet securely hold the collar 56 in each longitudinal station when the external force is removed.

One particular advantage of the indexing adapter 70 is that it can be installed on and removed from any osteotome 36 or drilling tool having at least one groove 45 in its shank 40 at a longitudinally coordinated location such that relative distance between the stop ring 58 and the apical end 48 will correspond to the intended drilling depth limit. Another advantage is that an adjustable position collar 56 can be utilized without forming multiple grooves in the tool shank 40, which would otherwise weaken the shank 40 with multiple stress-concentrating nodes that invite unwanted vibrations in use, and which add to manufacturing expense. And furthermore, multiple grooves in the tool shank 40 could increase the effort required post-operatively during the customary sterilization and cleaning processes.

The present invention, when configured with an indexing adapter 70, can be better suited to retrofit use across a wide range of drilling tools/burs marketed by different manufacturers. That is to say, in the case where manufacturers of different drilling tools form a groove 45 on their tool shank at different longitudinal positions relative to the apical end, or perhaps form grooves 45 of different shapes/sizes, it is possible to custom-manufacture an indexing adapter 70 for each manufacturer's specifications yet universally use the same collars 56 to fit across the spectrum of those various custom indexing adapters 70. For example, if Company X manufactures drilling tools and has a unique specification for the size and location of grooves 45 it forms on its tool shanks, and if Company Y manufactures drilling tools and has a consistently different specification for the size and location of grooves 45 it forms on its tool shanks, then an indexing adapter 70 specially fitting to Company X products can be offered, along with a different indexing adapter 70 specially fitting to Company Y products. And yet, the same collar 56 may be made to fit both types of indexing adapters 70.

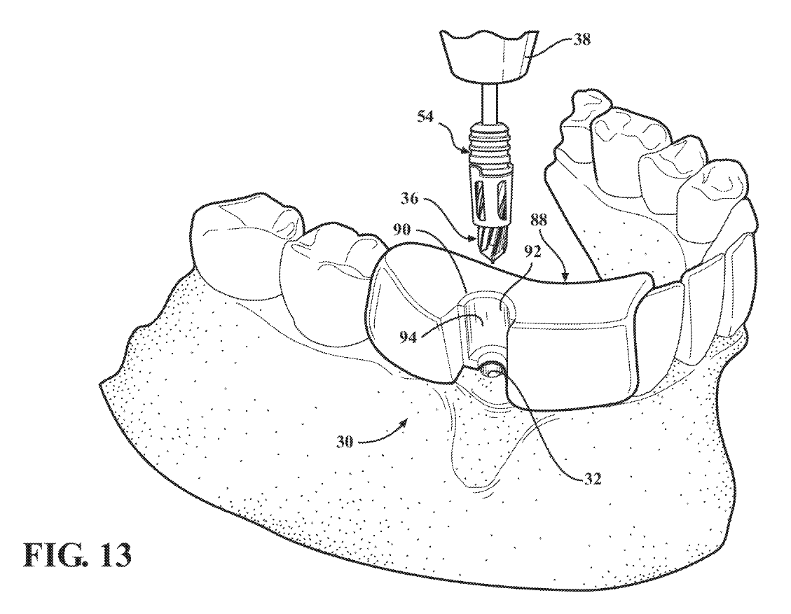

Turning now to FIGS. 13-16, the osteotome 36 and combined stop gauge 54 are show for use in an optional guided surgery application. Generally stated, guided surgery utilizes a custom-fabricated jig, generally indicated at 88, to provide pre-determined location and orientation assistance to the surgeon. The jig 88 may take many different forms, and is not intended to be limited to the illustrated examples which depict a fairly simple over-tooth guard-like structure. Indeed, the principles on this invention in the context of guided surgery applications, as described more fully below, can be implemented across many different platforms and types of jigs 88 including those jigs 88 which are larger, provide for many osteotomy 32 locations and/or are more complex in construction.

The jig 88 is configured to be secured over a target drilling location, which in the exemplary dental context may be an edentulous jaw site 30. The target drilling location will naturally vary for each patient and according to the needed surgical procedure. In order to best cooperate with the stop gauge 54, the jig 88 includes a novel guide bushing 90 which establishes an alignment valley 92 that is sized and shaped so as to center the longitudinal axis A of the osteotome 36 with the target drilling location when used in combination with the stop gauge 54. The alignment valley 92 may be formed in various ways. For example, in the illustrated examples the alignment valley 92 is formed in the shape of a semi-cylinder having an internal diameter that is configured slightly larger than the outer diameter of the collar 56 to receive the high-speed rotating collar 56 with minimal friction and yet without excessive play/clearance. Although not shown in the drawings, it is contemplated that the alignment valley 92 may take other forms including, for example, the shape of a “V” or a squared notch or other open geometry.

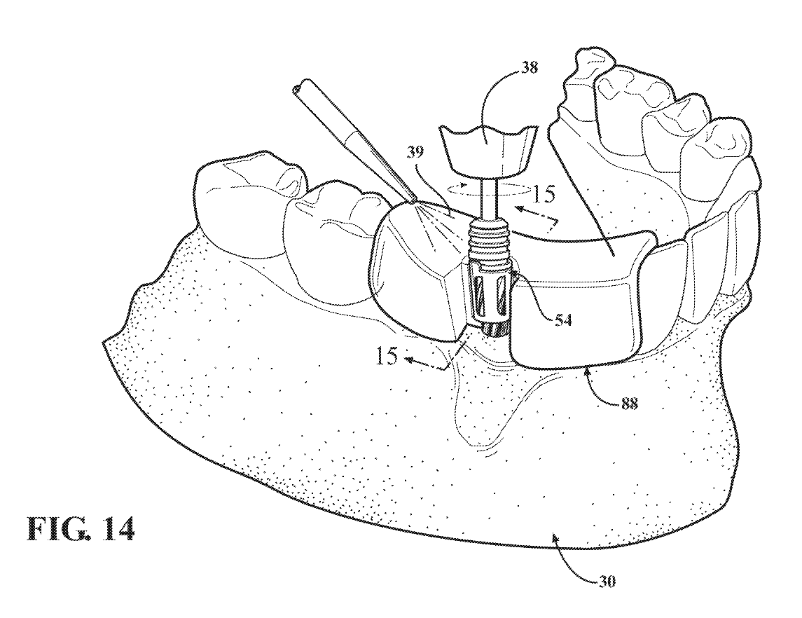

The alignment valley 92 is oriented within the jig 88 to open toward the outer gum of the patient, thus providing maximum access and visibility into the edentulous jaw site 30 for the surgeon. That is to say, the half-cylinder shape of the bushing 90 provides the operator with superior visual and physical access to the edentulous jaw site 30. The alignment valley 92, which is not fully enclosed like in many prior art designs, allows substantially increased irrigation capacity to the osteotomy 36, as illustrated in FIG. 14. Another advantage of the open (C-shaped) configuration of the alignment valley 92 is that, when used in conjunction with a rotary expanding osteotome 36, the bone is more freely able to expand laterally. Furthermore, the open-sided bushing 90 allows relatively long-length osteotomes 36 to be navigated laterally into position from a starting point outside the patient's mouth. Thus, for patients with small mouths, or with a condition that might otherwise make wide jaw opening uncomfortable, the semi-cylindrical bushing 90 offers a significant benefit. In one contemplated embodiment (not shown) the alignment valley 92 terminates directly adjacent the patient's skin or bone at the edentulous jaw site 30. In this manner, the aforementioned depth control afforded by the stop gauge 54 functions precisely in the manner described, with the alignment valley 92 providing a sighting-reference to aid in locating the osteotomy 32 and the stop gauge 54 providing depth control.

The alignment valley 92 may, optionally, include an internal ledge or abutment step 94. The abutment step 94 establishes an elevated surface configured to engage the spinning stop ring 58 of the collar 56 when the osteotome 36 has reached the desired drilling depth. In the case of a semi-cylindrical bushing 90, the abutment step 94 is semi-annular. One advantage of the abutment step 94 is to provide a perfectly smooth and perpendicular surface against which the rapidly rotating stop ring 58 will contact. Unlike the often imperfect surface of a patient's natural skin or exposed bone, the abutment step 94 is engineered to precision and will afford the surgeon certain and immediate haptic feedback when the desired drilling depth has been achieved. In cases where the collar 56 is made from a polymeric material, the smooth surface of the abutment step 94 may help avoid abrasion or distortion, thus extending the operating life of the stop gauge 54 and perhaps enabling re-use of the stop gauge 54 in one or more future surgical applications.

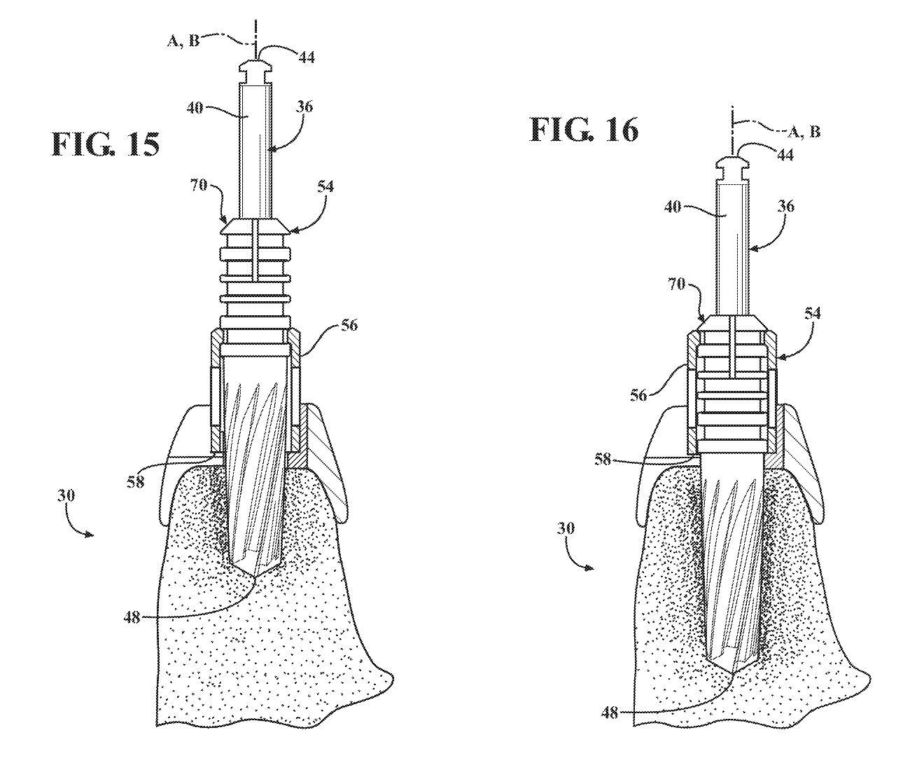

The elevation of the abutment step 94 above the patient's skin or bone at the edentulous jaw site 30 must be factored into the pre-set drilling depths established by the several longitudinal stations of the collar 56. For example, if the elevation of the abutment step 94 is 2 mm, then using the previous examples the actual drilling depths established by an indexing adapter 70 having five longitudinal stations will be 4, 6, 8, 9.5 and 11 mm, respectively. That is to say, the 2 mm elevation of the abutment step 94 (used as an example only) will subtract 2 mm from each of the otherwise predetermined drilling depths established by the indexing adapter 70 for the longitudinal stations of the collar 56. In FIG. 15, where the collar 56 is shown having been set to the first longitudinal station, the drilling depth will be (for example) 4 mm. However, in FIG. 16, the collar 56 is shown set to the fifth/last longitudinal station and the drilling depth will be (for example) 11 mm.

Of course, it is possible to design the indexing adapter 70 specifically for use with the guided surgical jig 88 so that the customary drilling depths are achieved without subtracting for the elevation of the annular abutment 94. Or alternatively, the depth number indicia may be designed to accommodate use of the stop gauge 54 with and without a jig 88 having an abutment step 94. For example, using the previous exemplary pre-set drilling depths, the numbers “6(4)” could be visibly embossed inside the first annular channel 80; the numbers “8(6)” in the second annular channel 80; the numbers “10(8)” in the third annular channel 80; the numbers “11.5(9.5)” in the fourth annular channel 80; and the numbers “13(11)” in the fifth/last annular channel 80. Of course, many alternatives are possible to accommodate the loss of drilling depth caused by the annular abutment 94.

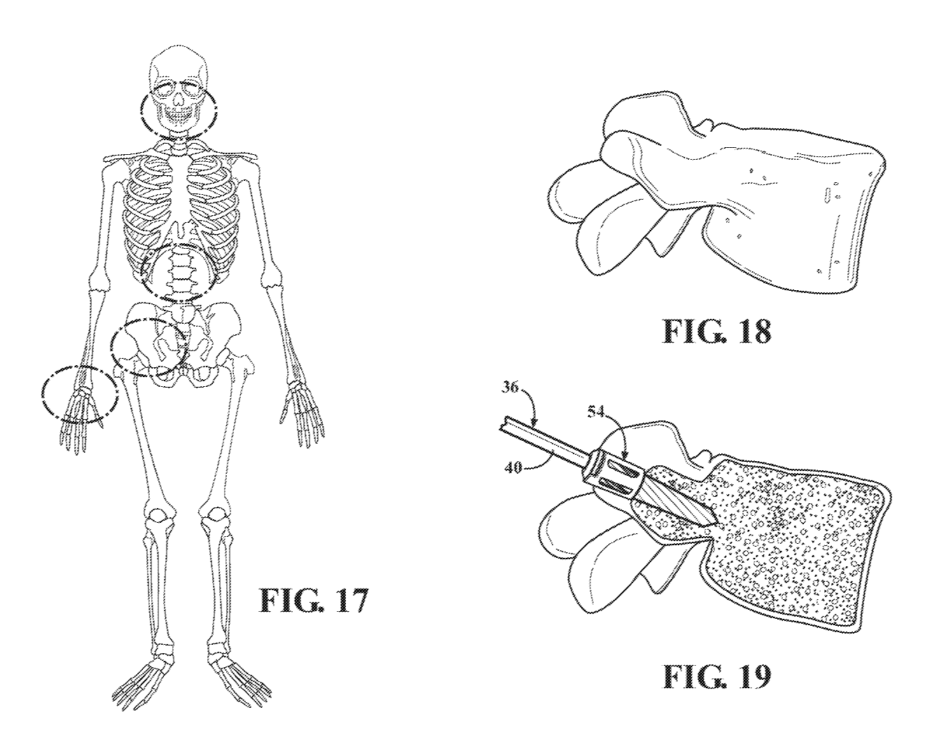

The novel features of this invention are not limited to dental applications, but in fact are directly adaptable to many orthopedic applications as well. FIG. 17 depicts a human skeleton, with but a few of the many possible zones of use being highlighted by broken circles. Indeed, the possible orthopedic applications are not limited to these highlighted zones only. Notwithstanding, one area of particular investigation is the spine or lumbar region, as exemplified in FIGS. 18 and 19. Spinal fusion, for example, is an orthopedic surgical technique that joins two or more vertebrae using a process called fixation which involves the placement of pedicle screws, rods, plates, or cages to stabilize the vertebrae and facilitate bone fusion. The autografting osteotome 36 is particularly well-suited to forming osteotomies in vertebrae to receive pedicle screws (not shown). A suitably-adapted stop gauge 54 can be used in conjunction with the osteotome 36, as shown in FIG. 19, to limit drilling depth according to a predetermined surgical protocol. Likewise, a suitably-adapted jig (not shown) can also be used in this lumbar application, as well as in other orthopedic applications. Furthermore, the concepts of this invention may be used to prepare holes in solid and cellular materials for industrial and commercial applications, such as in foamed metal or polymeric substrates, to name but a few.

The foregoing invention has been described in accordance with the relevant legal standards, thus the description is exemplary rather than limiting in nature. Variations and modifications to the disclosed embodiment may become apparent to those skilled in the art and fall within the scope of the invention. Furthermore, particular features of one embodiment can replace corresponding features in another embodiment or can supplement other embodiments unless otherwise indicated by the drawings or this specification.

What is claimed is:

1. An adjustable stop gauge for a bone drilling tool of the type having a body section and a shank joined in end-to-end fashion, said adjustable stop gauge comprising:
a tubular collar adapted to partially surround the body of a bone drilling tool, said collar defining a stop ring adapted to limit over-penetration of an apical tip of the body into bone, and
an indexing adapter connectable to the drilling tool and moveably supporting said collar, said indexing adapter configured to selectively locate said stop ring in any one of a plurality of longitudinal stations, wherein each station represents a different penetration depth of the drilling tool in the bone.

2. The adjustable stop gauge of claim 1, wherein said indexing adapter has a central bore adapted to mate with the shank of the drilling tool, said indexing adapter further including at least one cantilever locking segment, said locking segment including a spur extending inwardly from said central bore and adapted to engage within a groove of the drilling tool shank.

3. The adjustable stop gauge of claim 3, wherein said indexing adapter includes a plurality of annular channels each corresponding to a respective one of said longitudinal stations, each said channel adapted to selectively receive said inwardly extending barb of said collar.

4. The adjustable stop gauge of claim 1, wherein said collar includes a plurality of vane slots configured to permit the pass-through of irrigating fluid.

5. The adjustable stop gauge of claim 4, wherein said plurality of vane slots form an impeller capable of inwardly accelerating the flow of fluid, each pair of adjacent said vane slots being circumferentially separated by a respective longitudinally extending blade, each said blade having a pair of longitudinally extending edges defining a boundary with the adjacent respective said vane slots, at least one of said edges of each said blade being canted to impel irrigating fluid through said adjoining vane slot along a radially inward vector when said stop gauge is rotated.

6. A stop gauge for a bone drilling tool of the type having a body section and a shank joined in end-to-end fashion, said stop gauge comprising:
a tubular collar centered about a central axis, said collar adapted to partially surround the body of a drilling tool, said collar defining a stop ring adapted to limit over-penetration of an apical tip of the drilling tool into the bone, and
said collar including a plurality of vane slots configured to permit the pass-through of irrigating fluid.

7. The stop gauge of claim 6, wherein said plurality of vane slots form an impeller capable of inwardly accelerating the flow of fluid, said impeller comprises a plurality of vane slots, each pair of adjacent said vane slots being circumferentially separated by a respective longitudinally extending blade.

8. The stop gauge of claim 7, wherein said collar has an upper end opposite said stop ring, said collar having a lower annular cuff in the region between said stop ring and said vane slots and an annular upper cuff in the region between said upper end and said vane slots.

9. The stop gauge of claim 7, wherein said vane slots and said blades each extend in generally straight axial paths parallel to one another,

10. The stop gauge of claim 7, wherein each said blade has a pair of longitudinally extending edges defining a boundary with the adjacent respective said vane slots, at least one said edge of each said blade being canted to impel irrigating fluid through said adjoining vane slot along a radially inward vector when said stop gauge is rotated.

11. The stop gauge of claim 7, wherein each said blade has a pair of longitudinally extending edges defining a boundary with the adjacent respective said vane slots, both said edges of each said blade being canted in opposing directions to impel irrigating fluid through said respective adjoining vane slots along radially inward vectors when said stop gauge is rotated in either rotary direction.

12. The stop gauge of claim 6, further including an indexing adapter moveably supporting said collar, said indexing adapter configured to selectively locate said stop ring in any one of a plurality of longitudinal stations, wherein each station represents a different penetration depth of the drilling tool in the bone.

13. The stop gauge of claim 12, wherein said indexing adapter has a central bore adapted to mate with the shank of a drilling tool, said indexing adapter including at least one locking segment, said locking segment including a spur extending inwardly from said central bore and adapted to engage within an annular groove of a shank of the drilling tool, a plurality of annular channels each corresponding to a respective one of said longitudinal stations, each said channel adapted to selectively receive said inwardly extending barb of said collar.

14. A dental tool assembly for forming a hole of predetermined depth in bone, said assembly comprising:
a tubular collar adapted to partially surround the body of a bone drilling tool, said collar defining a stop ring adapted to limit over-penetration of an apical tip of the drilling tool into bone,
a jig configured to be secured relative to a target drilling location, said jig including a guide bushing, said guide bushing having a laterally open alignment valley adapted to receive said collar of said stop gauge, and
said alignment valley including an internal abutment step, said internal abutment step having a surface adapted to engage said stop ring of said collar when the apical tip of a drilling tool has reached a predetermined penetration limit in the bone.

15. The assembly of claim 14, wherein said alignment valley is generally semi-cylindrical and said abutment step is generally semi-annular, further including an indexing adapter connectable to the drilling tool and moveably supporting said collar between any one of a plurality of longitudinal stations, wherein each station represents a different penetration depth of the drilling tool in the bone, and wherein said collar includes a plurality of vane slots configured to permit the pass-through of irrigating fluid.
